(19) United States
(12) Patent Application Publication
(10) Pub. No.: US 2002/0102207 A1
(43) Pub. Date: Aug. 1, 2002

(54) Method of chemical marking for identifying fish stocks such as salmon using strontium chloride or hygroscopic compounds, thereof

(75) Inventors: Ronald A. Secor, Ferndale, WA (US); Leanne M. Secor, Ferndale, WA (US)

Correspondence Address: WILLIE KRAWITZ, 3001 CHAPEL HILL ROAD, ORANGE, CA, 92867, US

(73) Assignee: RONALD A. SECOR

(21) Appl. No.: 09/775,149

(22) Filed: Feb. 1, 2001

(51) Int. Cl. A61K 51/00

(52) U.S. Cl. 424/1.11; 424/667

(57) ABSTRACT

A method for chemically marking fish, and particularly salmon to determine migration patterns and spawning locations involves chemically marking salmon taken along select salmon origin, migration patterns or paths, and spawning areas. This avoids stress problems caused by current tagging, or physical marking of the salmon, and the detection system is less expensive and more accurate. Preferred chemical markers are strontium chloride and/or hygroscopic salts, thereof.

### BACKGROUND OF THE INVENTION

This invention involves a method for identifying different fish stocks, particularly salmon, to determine the origin, migration patterns and spawning locations.

One method of identifying salmon includes a thermal technique by an immersion in water at an elevated temperature, thereby producing a detectable signature in the skeletal structure of the fish. However, this method is both stressful and results in a deterioration in the skeletal structure of the fish.

Another method of identifying fish, particularly salmon involves the use of a coded wire implant in the sinus area of the fish which can be detected by a magnetometer of other detectors such as x-ray irradiation of the implanted tag. This method also causes the fish to undergo stress.

Still another method of identifying salmon involves removing the adipose fin, which is detected visually; however, all of the above methods are very expensive and time consuming; also, as indicated, these techniques tend to be stressful to the fish.

Still another method of identifying salmon involves treatment with tetracycline, however this treatment is beginning to receive criticism from various health organizations, and hence its use may be considerably reduced in the future.

Various publications describe methods for tagging fish, and these include U.S. Pat. Nos. 3,545,405; 4,392,236; 4,750,490; 4,743,193; 5,325,940; 6,020,143; and, SU 968,902 and SU 1,316,613. However, none of the above patents do not adequately address the above problems of fish tagging, particularly salmon tagging.

### THE INVENTION

According to the invention, there is provided a method of chemically marking the early life (or other) stage of the fish in the smolt stage with strontium chloride and/or hygroscopic salts thereof, or equivalents at different concentrations and multiple immersions, and then subsequently detecting the chemical signature in the odilif or skeletal structure using a scanning electron microscope, x-ray fluorescence, or by any other means.

The technique of the present invention avoids problems caused by other methods which may be more stressful to the fish, and the detection system is less expensive and requires much less time. Strontium chloride is used medically as a dental desensitizer, however, in view of its known toxicity, the concentration levels in use obviously should be maintained well below the levels which would become toxic to the fish. Hygroscopic compounds of SrCl₂ such as SrCl₂. 6H₂O may be used either alone or combined with SrCl₂, and these are considered the preferred embodiments.

Other strontium compounds may be considered which have reasonable water solubility and which may be incorporated into the skeletal structure or other parts of the fish, without affecting the long term health of the fish and also are detectable. These compounds might include strontium bromide, strontium bromate, strontium acetate, strontium nitrate, and mixtures thereof, provided these compounds do not adversely affect the fish. Moreover, a detection system may be employed not only to solely detect strontium itself, but also to detect individual components of a given strontium compound (e.g. halides, nitrates, acetate, etc.) as well as the specific strontium compound itself. Also, strontium isotopes may be employed, provided consumer safety conditions are maintained, i.e., the half life of the isotope is sufficiently low so that handling or consuming the fish will not pose a health problem.

### BACKGROUND OF THE INVENTION

This invention involves a method for identifying different fish stocks, particularly salmon, to determine the origin, migration patterns and spawning locations.

One method of identifying salmon includes a thermal technique by an immersion in water at an elevated temperature, thereby producing a detectable signature in the skeletal structure of the fish. However, this method is both stressful and results in a deterioration in the skeletal structure of the fish.

Another method of identifying fish, particularly salmon involves the use of a coded wire implant in the sinus area of the fish which can be detected by a magnetometer of other detectors such as x-ray irradiation of the implanted tag. This method also causes the fish to undergo stress.

Still another method of identifying salmon involves removing the adipose fin, which is detected visually; however, all of the above methods are very expensive and time consuming; also, as indicated, these techniques tend to be stressful to the fish.

Still another method of identifying salmon involves treatment with tetracycline, however this treatment is beginning to receive criticism from various health organizations, and hence its use may be considerably reduced in the future.

Various publications describe methods for tagging fish, and these include U.S. Pat. Nos. 3,545,405; 4,392,236; 4,750,490; 4,743,193; 5,325,940; 6,020,143; and, SU 968,902 and SU 1,316,613. However, none of the above patents do not adequately address the above problems of fish tagging, particularly salmon tagging.

### THE INVENTION

According to the invention, there is provided a method of chemically marking the early life (or other) stage of the fish in the smolt stage with strontium chloride and/or hygroscopic salts thereof, or equivalents at different concentrations and multiple immersions, and then subsequently detecting the chemical signature in the odilif or skeletal structure using a scanning electron microscope, x-ray fluorescence, or by any other means.

The technique of the present invention avoids problems caused by other methods which may be more stressful to the fish, and the detection system is less expensive and requires much less time.

1. A method for chemically marking fish, comprising immersion in water containing a water soluble strontium compound at a sufficient concentration level of the strontium compound. thereby incorporating strontium, strontium compound or component thereof, followed by detection at a suitable life cycle stage of the fish.

2. The method of claim 1, in which the water soluble strontium compound is selected from the class consisting of strontium chloride, hygroscopic compounds of strontium chloride, and mixtures thereof.

3. The method of claim 1, in which the water soluble compound of strontium is selected from the class consisting of strontium chloride, hygroscopic compounds of strontium chloride, strontium nitrate, strontium bromide, strontiumbromate, strontium acetate, and mixtures thereof.

4. The method of claim 1, in which the chemically marked fish is salmon.

5. The method of claim 1, in which the chemical marking takes place at multiple concentrations and multiple immersions.

6. The method of claim 1, in which the strontium compound is an isotope of strontium.
